(19) United States
(12) Patent Application Publication
(10) Pub. No.: US 2003/0185883 A1
(43) Pub. Date: Oct. 2, 2003

(54) Appetite suppressant for oral ingestion

(76) Inventor: Ronald S. Nietupski, Lockport, IL (US)

Correspondence Address: Todd S. Parkhurst, Holland & Knight LLP, Suite 800, 55 West Monroe Street, Chicago, IL, 60603-5144, US

(21) Appl. No.: 10/107,980

(22) Filed: Mar. 28, 2002

(51) Int. Cl. A61K 31/455; A61K 9/20

(52) U.S. Cl. 424/465; 514/354

(57) ABSTRACT

A method of suppressing human appetite, and a material therefore, are disclosed in the claims. The method includes the step of inducing body weight loss in a human by providing that human with material containing, as an active ingredient, 2-AcetylPyridine for human oral consumption. The material can be provided in the form of a lozenge. A related aspect of the invention relates to material for human ingestion to encourage body weight loss, the material comprising about 1% 2-AcetylPyridine volume/volume.

### BACKGROUND OF THE INVENTION

U.S. Pat. No. 4,521,427, entitled APPETITE SUPPRESSANT AND METHOD OF APPLICATION THEREOF describes and claims the use of 2-AcetylPyridine (2-AP™) in controlling human appetite for food. The present invention involves an improved method of delivering that active material to a human, and an improved delivery vehicle for that material.

### SUMMARY OF THE INVENTION

The improved method includes the step of inducing body weight loss in a human by providing that human with material containing, as an active ingredient, 2-AcetylPyridine for human oral consumption. The material can be provided in the form of a lozenge. A related aspect of the invention relates to material for human ingestion to encourage body weight loss, the material comprising about 1% 2-AcetylPyridine volume/volume.

### OBJECT OF THE INVENTION

Accordingly, it is a general object of this invention to provide, to the consumer, an appetite suppressant which is novel in nature, convenient and effective in use.

A more specific object is to provide such an appetite suppressant which can be used orally (ingested) as the method-of-delivery, utilizing both “retronasal olfaction” and “oral systemic absorption” mechanisms-of-action to maximize effectiveness.

An object of this invention is to improve the effectiveness of the action of 2-AP in appetite suppression.

Another object is to promote improved methods of delivering 2-AP to the human body.

A related object is to provide an oral lozenge for presenting or offering the appetite suppressant, thereby accomplishing previously stated objectives.

Other objects and advantages of the invention will become apparent by reference to the accompanying specification and claims.

### DESCRIPTION OF THE DRAWINGS

FIG. 1 is a histogram showing the distribution of weight loss results experienced during tests of the invention.

FIG. 2 is a bar chart comparing old and new methods of delivery of the appetite suppressant by average weight loss effectiveness.

FIG. 3 is a bar chart comparing old and new methods of delivery of the appetite suppressant by increase in total body weight loss effectiveness.

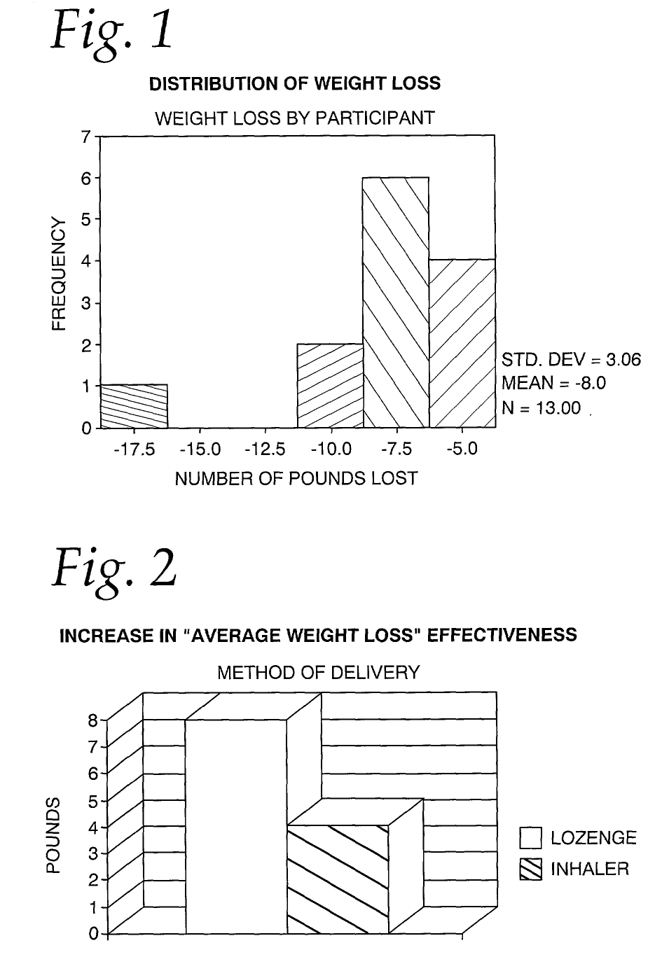

### DETAILED DESCRIPTION OF THE INVENTION

Briefly, and in accordance with the foregoing objects, a novel method for suppressing the appetite of an individual is described. This method comprises dissolving in the mouth (ingesting) a lozenge comprised of the combination of an effective amount of a volatile appetite suppressant so as to neutralize olfactory stimulus via chemosensory messaging and an effective amount of absorbable appetite suppressant to suppress appetite via oral systemic absorption.

Preferably the suppressant is an odorant having an intensity which matches 10-500 ppm (volume/volume of 1-butanol. It is currently standard procedure to match odor intensity to 1-butanol. This procedure is defined in ASTM E 544. The appetite suppressant presented herein was tested in accordance with ASTM E 544. I have found that odor intensities which match, preferably, 10-500 ppm (volume/volume) of 1-butanol are useful for suppressing appetite in humans. Odor intensities less than those matching 10 ppm of 1-butanol are generally too weak, except for very sensitive individuals. Odor intensities matching more than 500 ppm 1-butanol are quite strong and generally appear to be needed only by individuals who are rather insensitive to a particular suppressant.

Dieting aids are well known. While most are taken orally, most are ineffective under certain conditions because the suppressants used do not exhibit the characteristics and properties capable of utilizing the most effective “mechanisms-of-action” or have been banned because they were harmful to the user.

This invention is directed to a novel appetite suppressant. More particularly, this invention is directed to a novel method of applying the appetite suppressant in the form of an oral lozenge, capitalizing on “method-of-delivery” and “mechanisms-of-action” technologies improved over those of the appetite suppressant inhaler used as the former method of delivery disclosed in my U.S. Pat. No. 4,521,427, entitled APPETITE SUPPRESSANT AND METHOD OF APPLICATION THEREOF.

A standardized clinical study was conducted and subsequently analyzed using an ANALYSIS OF VARIANCE FOR REPEATED MEASURES, which is one of the most powerful and efficient research designs for time series data in which a sample is provided with a baseline measure and a final outcome measure. The initial sample contained 15 participants of which 13 completed the study tasks. All participants were provided with lozenges containing the active ingredient 2-AP in a concentration the equivalent of a 1% volume/volume of 1-butanol which is equal to that of the study cited in my U.S. Pat. No. 4,521,427.

However, in order to elicit an increase in effectiveness utilizing the combination of the retronasal olfaction and oral systemic absorption mechanisms inherent of the oral/ingested (lozenge) delivery system and attain a desired 5-8 minute dissolution time utilizing this previously patented formulation, the lozenge size (through “trial and error”) was determined to be 1.5 grams. Excipients such as mannitol and/or sorbitol may be used to produce additional desirous results such as a “sugar-free” product. Use of other excipients, diluents or solvents, to achieve similar or other goals, may be effective as well.

Participants were instructed to consume as many as deemed necessary throughout the day. All participants were instructed not to participate in any exercise program for the duration of the 14 day study, nor was any diet regimen imposed or psychological reinforcement availed.

Specifically, all participants were weighed at the beginning of the study and at its completion. Per accepted protocol and standards, the 95% confidence level was used to test statistical significance. The 95% confidence level is used in many sensory evaluations by medical and psychological studies, and is considered sufficient by experts in medicine and psychology for supporting claims to noted differences. The results, after the 14 day study, indicated that the average weight loss per participant was 7.96 lbs. which equated to a 3.2% loss of total body weight. See Table 1.

TABLE 1
Average Weight of Participants Average Weight
Loss, & Sample Variability

| | | | | | Std. |
|---|---|---|---|---|---|
| | N | Minimum | Maximum | Mean | Deviation |
| Start Weight of | 13 | 160 | 338 | 255.58 | 57.84 |
| Participant | | | | | |
| End Weight of | 13 | 155 | 328 | 247.62 | 56.70 |
| Participant | | | | | |
| Weight Difference | 13 | −17.00 | −5.00 | −7.9615 | 3.0582 |
| Valid N (listwise) | 13 | | | | |

This result was statistically significant, F(1,12)=88.11, p=0.00, indicating that the lozenges were highly effective in reducing one's body weight. Not only was the treatment highly successful, but the validity of these results is more than 95% certain. See Table 2.

TABLE 2
Effect of Lozenge in Body Weight Reduction Over Time

| Measure: | | Type III | | | | | | |
|---|---|---|---|---|---|---|---|---|
| MEASURE_1 | | Sum of | | Mean | | | Noncent. | Observed |
| Source | TIME | Squares | df | Square | F | Sig. | Parameter | Power^{a} |
| TIME | Linear | 412.010 | 1 | 412.010 | 88.106 | .000 | 88.106 | 1.000 |
| Error | Linear | 56.115 | 12 | 4.676 | | | | |
| (TIME) | | | | | | | | |

^{a.}Computed using alpha = .05

So that the distribution of weight loss could readily be seen visually, a histogram was constructed. Significantly, the minimum amount of weight loss was 5 lbs., and the maximum weight loss was 17 lbs.

Lozenge vs. Inhaler Effectiveness

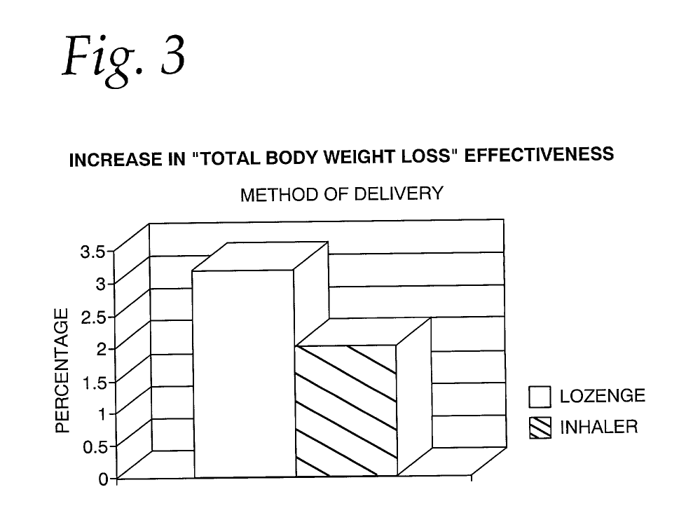

In comparing these results to those of the previous U.S. Pat. No. 4,521,427 test results, in which participants lost an average of 4.14 lbs. in 14 days, or an average 2.07% total body weight loss (as compared to 7.96 lbs. and 3.20% respectively), it is readily seen that the new Method-of-Delivery with its associated Mechanisms-of-Action is significantly and surprisingly more successful. See FIGS. 2 and 3.

The average weight loss per participant was nearly double and the average total body weight loss was 55% more effective.

In summary, both the primary and secondary objectives of the study were satisfied. While it is apparent that the lozenge treatment is highly effective in losing total body weight, the oral/ingestion lozenge method-of-delivery was proven significantly more effective than the nasal inhaler method-of-application previously patented.

What has been described herein is a novel appetite suppressant, and a novel method of delivery.

While the suppressant of the present invention has been described with reference to a preferred “oral/ingestion” method-of-delivery thereof, the invention is not limited to a specific vehicle such as a lozenge. On the contrary, alternatives (i.e., gums, pills, granules, tabs, mints, baked/frozen goods, powders, wafers, liquids, etc.), and other changes or modifications may become apparent to those skilled in the art upon reading the foregoing description. Accordingly, such alternatives, changes or modifications are to be considered as forming a part of the invention insofar as they fall within the spirit and scope of the appended claims.

1. A method of inducing body weight loss in a human comprising the steps of forming material for oral ingestion containing, as an active ingredient, 2-AcetylePyridine; and providing said material to the human for oral consumption.

2. A method according to claim 1 comprising the step of providing as much material to the human as the human desires to consume.

3. A method according to claim 1 wherein the step of forming material for oral ingestion includes the step of providing about one percent 2-AcetylePyridine volume/volume in said material.

4. A method of inducing body weight loss in a human comprising the steps of forming lozenges containing, as an active ingredient, 2-AcetylePyridine and providing said lozenges to the human for oral consumption.

5. A method according to claim 4 including the step of providing as many lozenges to the human as said human desires to consume.

6. A method according to claim 4 wherein the step of forming the lozenge for oral ingestion includes the step of providing about one percent 2-AcetylePyridine volume/volume in said lozenge.

7. A material for human ingestion to encourage body weight loss in the human, the material comprising about one percent 2-AcetylePyridine volume/volume.

8. A lozenge for human ingestion to encourage body weight loss in the human, the lozenge comprising about one percent 2-AcetylePyridine volume/volume.
